(19) United States
(12) Patent Application Publication
(10) Pub. No.: US 2026/0094683 A1
(43) Pub. Date: Apr. 2, 2026

(54) METHOD AND DEVICE FOR ADMINISTERING FUNCTIONAL BEHAVIOR ASSESSMENT AND GENERATING POSITIVE BEHAVIOR SUPPORT STRATEGY, AND NON-TRANSITORY COMPUTER-READABLE RECORDING MEDIUM

(71) Applicant: National Taiwan Normal University, Taipei City (TW)

(72) Inventors: Hui-Ting WANG, Taipei City (TW); Jia-Yin WANG, Taoyuan City (TW); Fang-Yu LIN, Taichung CIty (TW); Jia-Chi LIANG, Taoyuan City (TW)

(21) Appl. No.: 19/174,177

(22) Filed: Apr. 9, 2025

(30) Foreign Application Priority Data
Sep. 30, 2024 (TW) 113137257

(51) Int. Cl. G16H 20/00; G06F 3/0482; G06F 3/0483; G06F 3/04845; G16H 10/20

(52) U.S. Cl. CPC G16H 20/00; G06F 3/0482; G06F 3/0483; G06F 3/04845; G16H 10/20

(57) ABSTRACT

A method for administering a functional behavior assessment (FBA) and generating a positive behavior support (PBS) strategy. The method is implemented by a computer device and includes steps of: displaying a behavior problem collection page for inputting of a target behavior problem of a client; based on the target behavior problem thus inputted, generating and displaying a questionnaire to be completed; based on a response to the questionnaire, determining probable function of the target behavior problem of the client; based on the response to the questionnaire, the probable function of the target behavior problem, and logic of the competing behavior pathway model, generating and displaying at least one PBS strategy for selection; and based on a selected PBS strategy of the at least one PBS strategy, generating and displaying a lesson plan that corresponds to the selected PBS strategy.

### CROSS-REFERENCE TO RELATED APPLICATION

This application claims priority to Taiwanese Invention Patent Application No. 113137257, filed on Sep. 30, 2024, the entire disclosure of which is incorporated by reference herein.

### FIELD

The disclosure relates to a method and a device for administering a functional behavior assessment (FBA) and generating an intervention plan, and more particularly to a method and device for assessing behavior problems and automatically generating a corresponding positive behavior support (PBS) strategy and lesson plan.

### BACKGROUND

In order to assist clients with emotional and behavioral needs to participate in life and integrate into society, special education personnel need to assess behavior problems of clients to formulate corresponding lesson plans. However, in order to evaluate clients with emotional and behavioral needs, in addition to the assessments involving complicated information, the data collection process is relatively difficult and time-consuming, and may not yield accurate results. This could result in a lack of accuracy of an assessment, causing the formulated lesson plan to fail to meet the needs of the client. Furthermore, there is often a shortage and high turnover rate of special education personnel, resulting in poor personnel training and affecting the development of lesson plans.

### SUMMARY

Therefore, an object of the disclosure is to provide a method and a device for administering a functional behavior assessment (FBA) and generating a positive behavior support (PBS) strategy that can alleviate at least one of the drawbacks of the prior art, and a non-transitory computer-readable recording medium for storing instructions to carry out the method.

According to an aspect of the disclosure, a method for administering an FBA and generating a PBS strategy is to be implemented by a computer device and includes steps of:
displaying a behavior problem collection page for inputting of a target behavior problem of a client;
based on the target behavior problem thus inputted, generating and displaying a questionnaire to be completed;
based on a response to the questionnaire, determining a probable function of the target behavior problem of the client;
based on the response to the questionnaire, the probable function of the target behavior problem, and logic of a competing behavior pathway model, generating and displaying at least one PBS strategy for selection; and
based on a selected PBS strategy of the at least one PBS strategy, generating and displaying a lesson plan that corresponds to the selected PBS strategy.

According to another aspect of the disclosure, a device for administering a functional behavior assessment (FBA) and generating a PBS strategy includes a display unit, an input unit, and a processing unit electrically connected to the display unit and the input unit. The processing unit is configured to make the display unit display a behavior problem collection page for inputting of a target behavior problem of a client via the input unit. Based on the target behavior problem thus inputted, the processing unit is configured to generate and make the display unit display a questionnaire to be completed via the input unit, wherein the questionnaire includes questions related to the target behavior problem thus inputted. Based on a response to the questionnaire, the processing unit is configured to determine a probable function of the target behavior problem of the client. Then, based on the response to the questionnaire, the probable function of the target behavior problem and logic of a competing behavior pathway model, the processing unit is configured to generate at least one PBS strategy and make the display unit display the at least one PBS strategy for selection via the input unit. Based on a selected PBS strategy of the at least one PBS strategy, the processing unit is configured to generate a lesson plan that corresponds to the selected PBS strategy and make the display unit display the lesson plan.

According to still another aspect of the disclosure, a non-transitory computer-readable recording medium stores a software program that, when installed and executed by a computer device, enables the computer device to implement the aforementioned method for administering an FBA and generating a PBS strategy.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a flow chart illustrating a method for administering a functional behavior assessment (FBA) and generating a positive behavior support (PBS) strategy according to an embodiment of the disclosure.

FIG. 2 is a block diagram illustrating a device for administering an FBA and generating a PBS strategy according to an embodiment of the disclosure.

FIG. 3 illustrates a behavior problem collection page according to an embodiment of the disclosure.

FIG. 4 illustrates a questionnaire with detailed questions about an inputted target behavior problem of throwing objects according to an embodiment of the disclosure.

FIG. 5 illustrates a Functional Assessment Screening Tool (FAST) questionnaire according to an embodiment of the disclosure.

FIG. 6 illustrates a Questions about Behavioral Function (QABF) questionnaire according to an embodiment of the disclosure.

FIG. 7 illustrates a results page of an FBA according to an embodiment of the disclosure.

FIG. 8 illustrates a strategy generation page according to an embodiment of the disclosure.

FIG. 9 illustrates a strategy selection page according to an embodiment of the disclosure.

FIG. 10 illustrates contents of a lesson plan according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

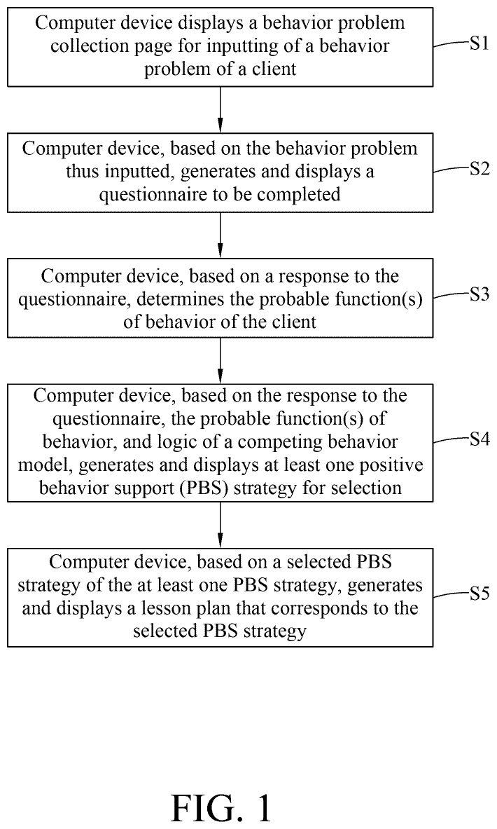

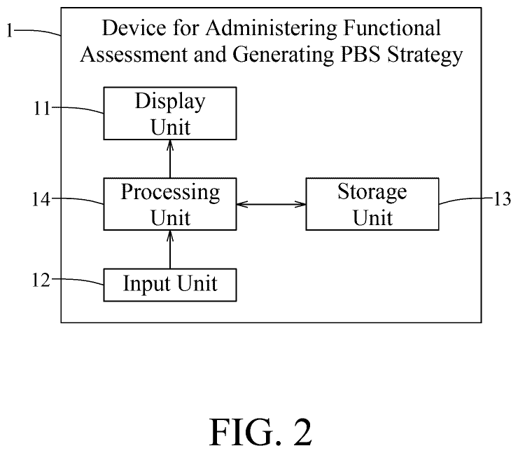

FIG. 1 is a flow chart that illustrates a method for administering a functional behavior assessment (FBA) and generating a positive behavior support (PBS) strategy according to an embodiment of the disclosure. The method is to be implemented by a device for administering an FBA and generating a PBS strategy 1 (hereinafter device 1, see FIG. 2). The device 1 is a computer device, for example, but not limited to, a personal computer, a laptop, a tablet, a smartphone, etc. The device 1 includes a display unit 11, an input unit 12, a storage unit 13, and a processing unit 14 electrically connected to the display unit 11, the input unit 12 and the storage unit 13.

The display unit 11 may be embodied using, for example, but not limited to, a flat panel display. The input unit 12 may be embodied using, for example, a physical keyboard and mouse, or a transparent touch panel disposed on the display unit 11 (flat panel display), which together with the display unit 11 forms a touch display, but the disclosure is not limited thereto. The storage unit 13 may be embodied using a memory device or memory module of any form. The processing unit 14 may be embodied using a processor that has computational capabilities, such as a central processing unit (CPU). The processing unit 14 is able to install and execute a software program stored on a non-transitory computer-readable recording medium (such as the storage unit 13), so as to implement the method illustrated in FIG. 1.

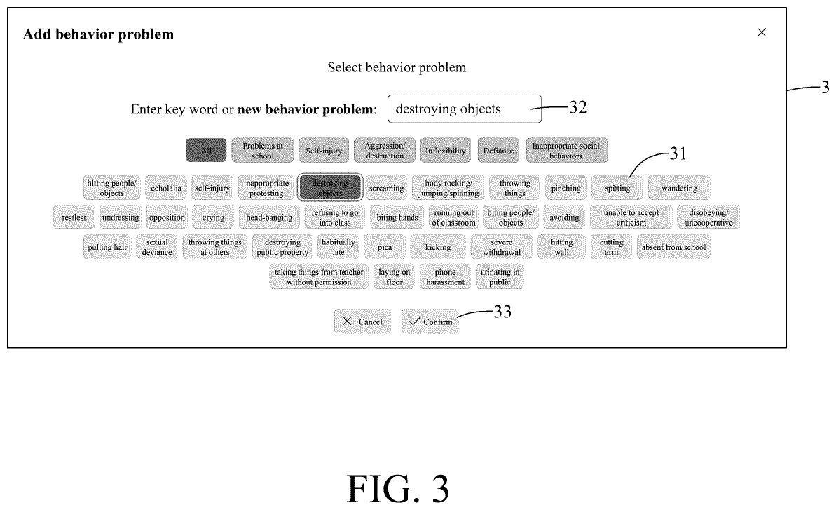

As shown in step S1 in FIG. 1, after the processing unit 14 executes the software program, the processing unit 14 makes the display unit 11 display a behavior problem collection page 3 (see FIG. 3) for inputting of a plurality of target behavior problems of a client (e.g., a client named James) via the input unit 12. More specifically, the behavior problem collection page 3 displays a plurality of behavior problems 31 for selection, and displays an input field 32 for manual entry of a target behavior problem. That is, when a behavior problem that a user wants to input is not included in the plurality of behavior problems 31 displayed on the display unit 11, the user can manually enter a behavior problem into the input field 32, and the processing unit 14 will make the manually entered behavior problem serve as one of the plurality of behavior problems thus selected (hereinafter referred to as selected behavior problems). Furthermore, the user can, via the input unit 12, drag and reorder the selected behavior problems according to severity levels thereof, for example, by placing more severe selected behavior problems at the top and placing less severe selected behavior problems at the bottom, to assist the user in identifying a behavior problem that needs to be addressed first. The processing unit 14 then enables the selected behavior problems to serve as the plurality of target behavior problems thus inputted.

Then, after the user confirms the selected behavior problems (e.g., by selecting a confirm button 33 on the behavior problem collection page 3), the processing unit 14 generates a questionnaire and makes the display unit 11 display the questionnaire for the user to complete (step S2 in FIG. 1). In one embodiment, the processing unit 14 reads a questionnaire from the storage unit 13 and controls the display unit 11 to display the questionnaire.

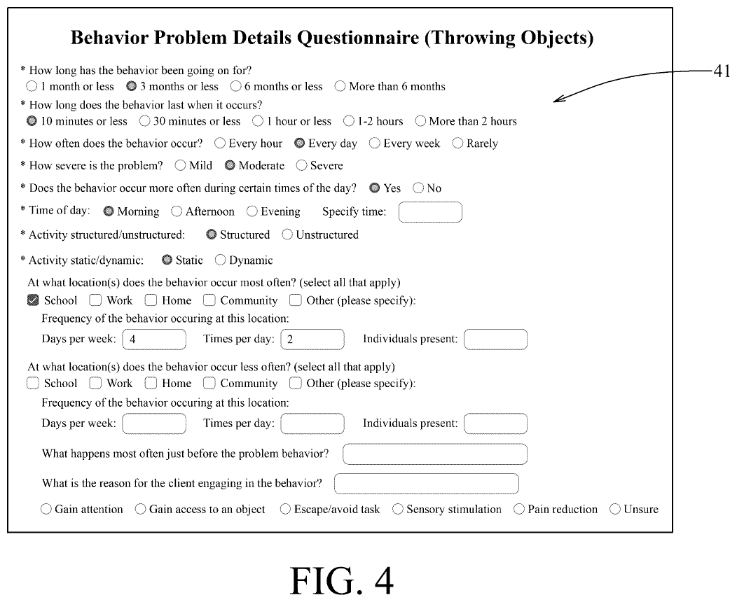

Specifically, as shown in FIG. 4, the questionnaire includes detailed questions 41 related to one or more of the plurality of target behavior problems thus inputted. The following discussion will use the target behavior problem of throwing objects as an example to illustrate the method of this embodiment. FIG. 4 shows an example of the detailed questions 41 directed to the target behavior problem of throwing objects, but the disclosure is not limited thereto. The user replies, via the input unit 12, to the detailed questions 41, which are typically multiple choice, yes/no or short answer questions. In one embodiment, the processing unit 14 generates a plurality of questionnaires that respectively include the plurality of target behavior problems thus inputted.

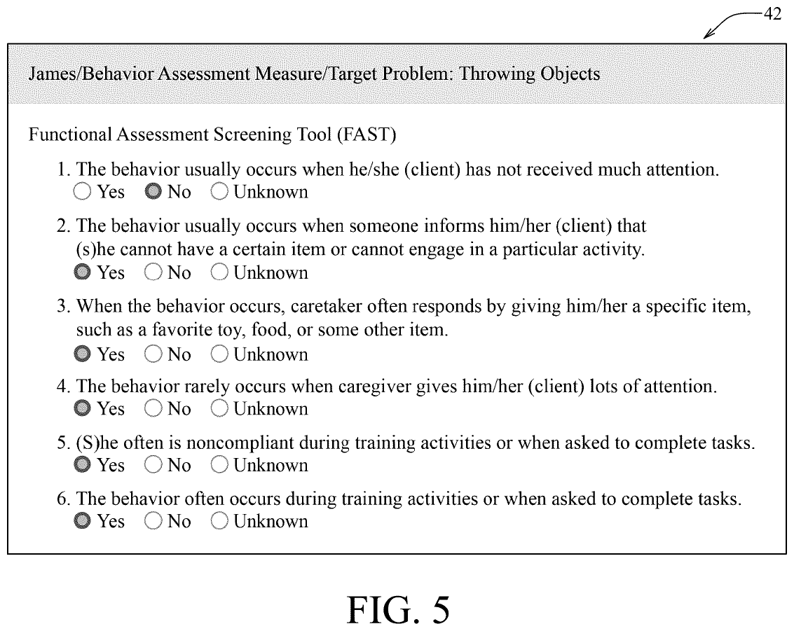

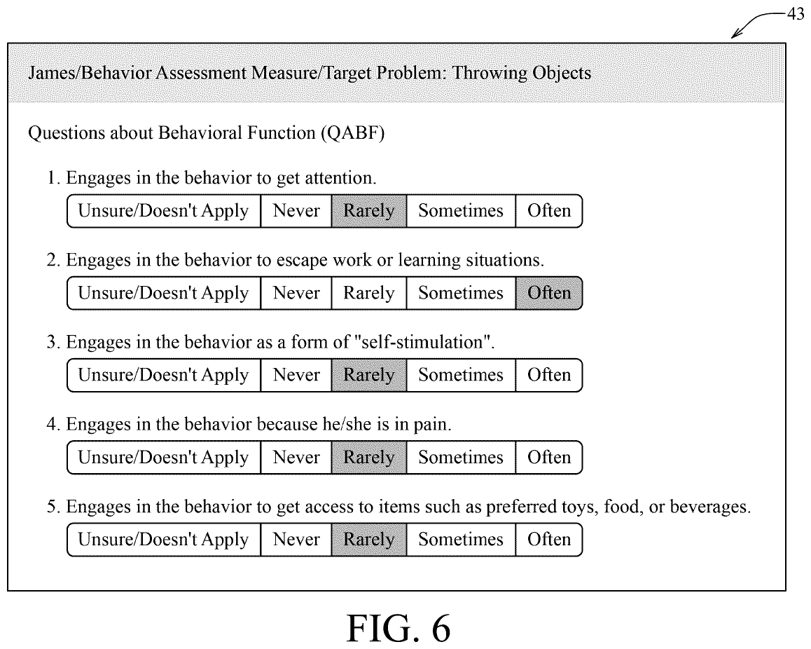

At this time, if the processing unit 14 determines that a reply to a questionnaire question (that is, one of the detailed questions 41) about the reason for engaging in a target behavior problem (e.g., “What is the reason for the client engaging in the behavior?”) is inputted as unsure, the processing unit 14 makes the questionnaire further include a first behavior evaluation form and a second behavior evaluation form for the user to complete. According to a recent publication, two assessment measures, Functional Analysis Screening Tool (FAST) and Questions About Behavior Function (QABF), when used in combination with each other, can improve accuracy of an FBA to 80% or higher (Romani, P. W., Luehring, M.C., Hays, T.M., & Boorse, A.L. (2023). Comparisons of functional behavior assessment procedures to the functional analysis of problem behavior. Behavior Analysis: Research and Practice, 23(1), 36-48. https://doi.org/10.1037/bar0000258). According to the embodiment of the disclosure, the first behavior evaluation form may be embodied using FAST 42 (see FIG. 5), and the second behavior evaluation form may be embodied using QABF 43 (see FIG. 6), but the disclosure is not limited thereto. After the user replies, via the input unit 12, to the detailed questions 41, the FAST 42 and the QABF 43, a response to the questionnaire is sent to the processing unit 14.

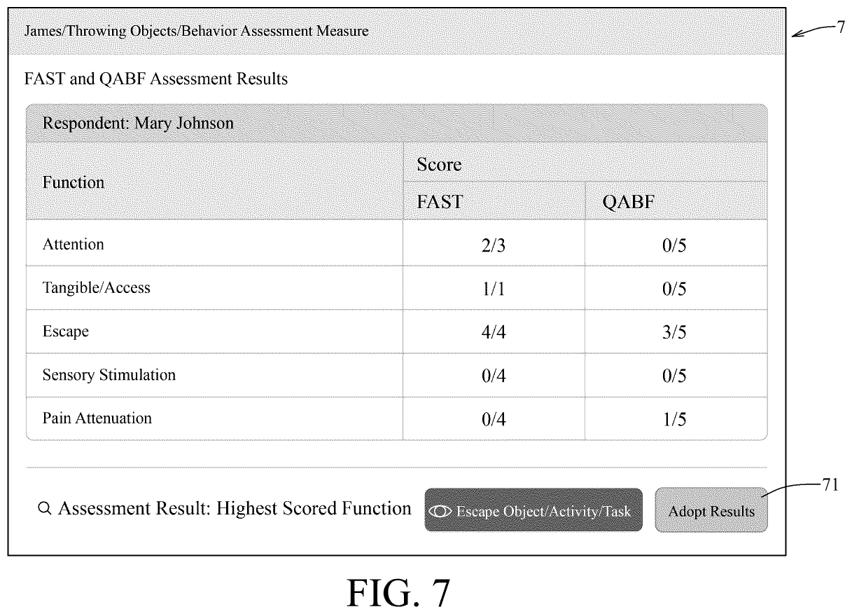

Then, as shown in step S3 in FIG. 1, the processing unit 14, based on the response to the questionnaire (including a response to the FAST 42 and a response to the QABF 43), automatically performs a cross-comparison to determine probable function(s) of the target behavior problem of the client and generates an FBA results page 7 (see FIG. 7) which displays the probable function(s) of the target behavior problem of the client (e.g., escape object/activity/task).

Specifically, the processing unit 14 utilizes a decision-tree algorithm to perform the cross-comparison on the response to the FAST 42 and the response to the QABF 43 to determine the probable function(s) of the target behavior problem, wherein the determination is made based on foundational principles including:
a. in a case where the response to the FAST 42 and the response to the QABF 43 indicate a same function of the target behavior problem, giving the function of the target behavior problem a highest rating, and if response rates to questions related to that function of the target behavior problem on the FAST 42 and on the QABF 43 exceed 50%, determining that function of the target behavior problem (i.e., the function of the target behavior problem with the highest rating) to be the probable function of the target behavior problem of the client;
b. in a case where the response to the FAST 42 and the response to the QABF 43 indicate multiple functions of the target behavior problem, and the response rates to questions related to two of the multiple functions of the target behavior problem on the FAST 42 and on the QABF 43 exceed 50%, determining the two functions of the target behavior problem to be the probable functions of the target behavior problem of the client; and
c. in a case other than the abovementioned principles a. and b., recommending that the FAST 42 and the QABF 43 be completed again (e.g., having another person familiar with the client complete the FAST 42 and the QABF 43), and performing a cross-comparison on a new response to the FAST 42 and a new response to the QABF 43.

On the other hand, if it is determined that a reply to the detailed questions 41 about the reason for engaging in a target behavior problem is inputted as known (e.g., by selecting from a selection of functions of the target behavior problem), the user replies to only the detailed questions 41 and submits a response to the processing unit 14, and in step S3 mentioned above, the processing unit 14 determines the probable function(s) of the target behavior problem of the client (e.g., escape object/activity/task) based on the response to the detailed questions 41.

Then, after the user adopts the probable function(s) of the target behavior problem determined by the processing unit 14, such as by selecting an adopt results button 71 on the FBA results page 7 or by selecting an adopt option (not shown in drawings) that corresponds to the probable function(s) of the target behavior problem directly determined by the processing unit 14 based on the response to the detailed questions 41, the method flow proceeds to step S4 of FIG. 1.

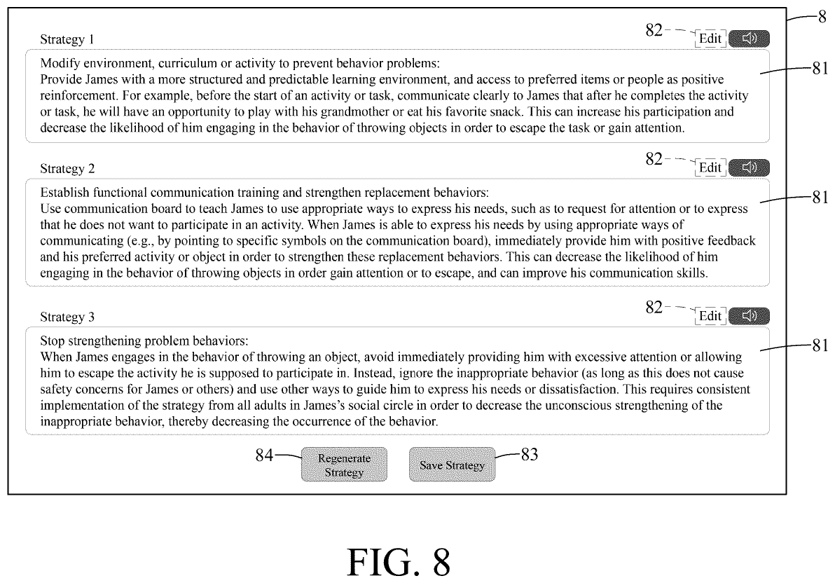

As shown in step S4, the processing unit 14, based on the response to the questionnaire, the probable function(s) of the target behavior problem (e.g., escape object/activity/task) and logic of a competing behavior pathway model, immediately generates and makes the display unit 11 display a strategy generation page 8 (see FIG. 8) that includes at least one PBS strategy for the user to review. In the embodiment of the disclosure, the generation of three PBS strategies is used for illustration purposes, including strategy 1 (modify environment, curriculum or activity to prevent behavior problems), strategy 2 (establish functional communication training and reinforce replacement behaviors) and strategy 3 (withholding reinforcement for the problem behaviors).

The competing behavior pathway model mentioned above refers to the competing behavior pathway developed by the fields of applied behavior analysis and PBS. The competing behavior pathway model is a framework used to link FBA to behavior support plans and strategies. By identifying the function of the target behavior problem and finding a “competing” positive behavior that serves the same function, this framework can suggest a more appropriate alternative behavior and strategies to replace the target behavior problem and maintain the same consequence (i.e., response, action, or event that follows a behavior).

Specifically, as shown in FIG. 8, the strategy generation page 8 includes, with respect to each of the PBS strategies (strategy 1, strategy 2, strategy 3), a field 81 that displays contents of the PBS strategy, and an edit button 82. In response to the processing unit 14 determining that the edit button 82 is selected, the processing unit 14 makes editable the field 81 so as to allow the user to edit the contents of the corresponding PBS strategy via the input unit 12.

The strategy generation page 8 further includes a save strategy button 83 and a regenerate strategy button 84. In response to the processing unit 14 determining that the save strategy button 83 is selected, the processing unit 14 saves to the storage unit 13 the contents of each of the PBS strategies (strategy 1, strategy 2, strategy 3). In response to the processing unit 14 determining that the regenerate strategy button 84 is selected, the processing unit 14 regenerates the contents of each of the PBS strategies (strategy 1, strategy 2, strategy 3).

It is worth noting that the processing unit 14 in the embodiment of the disclosure utilizes an online generative artificial intelligence (AI) model based on a first prompt, the response to the questionnaire, the probable function(s) of the target behavior problem, and the logic of the competing behavior pathway model to immediately generate the contents of each of the PBS strategies. The first prompt prompts the generative AI model to generate and compose a paragraph based on three prompt structures (role setting, task description, output template), the response to the questionnaire, and the probable function(s) of the target behavior problem, wherein the prompt structures are as follows:
Role setting: behavior analyst,
Task description: Develop strategies based on competing behavior pathway model,
Output template: Strategy 1. xxxxx: Strategy 2. xxxxx: Strategy 3. xxxxx:.

Based on the above information, the generative AI model generates three PBS strategies as shown in FIG. 8. For privacy protection, a client name on the response to the questionnaire is first converted into a default placeholder name (e.g., John Doe) by the software program before being provided to the generative AI model, then the software program converts the default placeholder name (e.g., John Doe) shown in the PBS strategies generated by the generative AI model back to the client name.

For example, the response to the questionnaire (and information about the client) provided to the generative AI model may be as follows:
John Doe/male/4 years old/mild physical and mental disabilities/Autism (ICF: b122) /Needs: communication support, emotional behavioral support/Preferred object: toy cars/ Preferred person: unsure/Target behavior problem: throwing objects/Function(s) of target behavior problem: escape object/activity/task/Frequency: every day/Severity: moderate/ Time of day during which target behavior problem often occurs: morning/Location at which behavior problem often occurs: school.

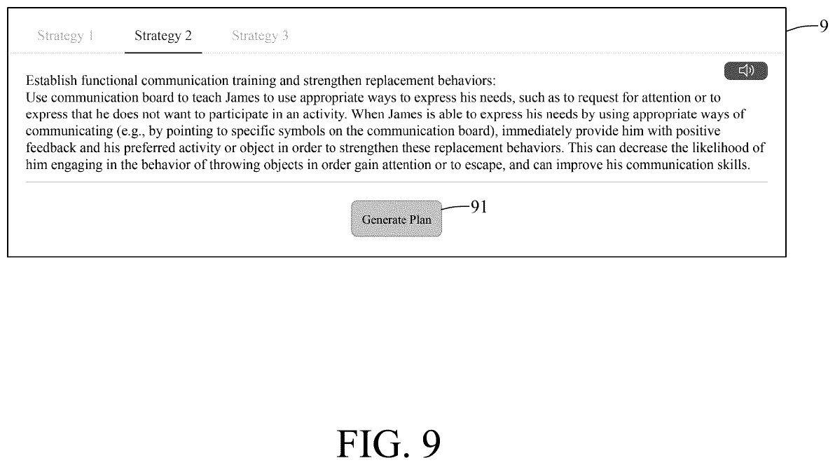

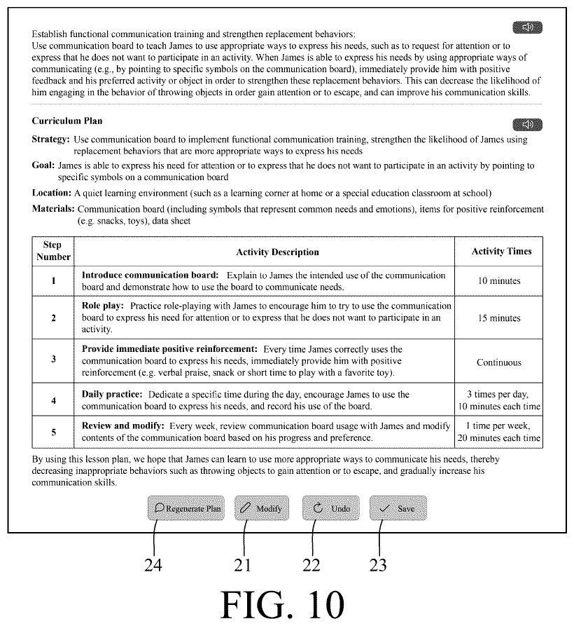

After the processing unit 14 saves the contents of each of the PBS strategies (strategy 1, strategy 2, strategy 3), the processing unit 14 makes the display unit 11 display a strategy selection page 9 (see. FIG. 9) that includes the three PBS strategies (strategy 1, strategy 2, strategy 3) for the user to select. At this time, the user can review the contents of each PBS strategy by selecting the corresponding PBS strategy. When the user wants to obtain a lesson plan that corresponds to the currently-reviewed PBS strategy and selects a generate plan button 91 on the strategy generation page 9, the processing unit 14 determines that the PBS strategy that was being reviewed when the generate plan button 91 was selected has been selected, and, as shown in step S5 in FIG. 1, the processing unit 14 generates a lesson plan (see FIG. 10) that corresponds to the selected PBS strategy (e.g., strategy 2) and makes the display unit 11 display contents of the lesson plan.

As shown in FIG. 10, the lesson plan includes a curriculum plan, a list of activity steps and activity times (durations and/or frequencies) corresponding to the activity steps, respectively, a modify button 21, an undo button 22, a save button 23, and a regenerate plan button 24. In response to the modify button 21 being selected, the processing unit 14 allows the user to, via the input unit 12, edit the curriculum plan, the list of activity steps and the corresponding activity times. In response to the undo button 22 being selected, the processing unit 14 restores to an unmodified version the edited curriculum plan, the edited list of activity steps and the corresponding edited activity times. In response to the save button 23 being selected, the processing unit 14 saves to the storage unit 13 the curriculum plan, the list of activity steps and the corresponding activity times of the lesson plan (original or edited version). In response to the regenerate plan button 24 being selected, the processing unit 14 regenerates a lesson plan based on the contents of the selected PBS strategy.

It is worth noting that the processing unit 14 utilizes a generative AI model based on a second prompt and contents of the selected PBS strategy to immediately generate the corresponding lesson plan. The second prompt prompts the generative AI model to generate and compose a paragraph based on three prompt structures (role setting, task description, output template) and the contents of the selected PBS strategy (e.g., strategy 2), wherein the prompt structures are as follows:
Role setting: behavior analyst,
Task description: Based on strategy provided, generate contents of required items of a lesson plan corresponding to the strategy,
Output template: Use Markdown syntax to output in a table format the contents of specified items such as required materials.

Based on the above information, the generative AI model generates a lesson plan corresponding to the selected PBS strategy (e.g., strategy 2), as shown in FIG. 10. For privacy protection, when the contents of the selected PBS strategy contain the client name, the client name referenced in the PBS strategy is first converted into a default placeholder name (e.g., John Doe) by the software program before being provided to the generative AI model, and then the software program converts the default placeholder name (e.g., John Doe) shown in the contents of the lesson plan generated by the generative AI model back to the client name. In addition, the abovementioned generative AI model may be, for example, GPT-4o or GPT-4o-mini, but the disclosure is not limited to a specific type of generative AI.

In summary, the aforementioned embodiment is relevant to the fields of applied behavior analysis, augmentative and alternative communication, and social work, and integrates interdisciplinary empirical theories and practical experiences in the design of an input interface for the user to easily and quickly input information to describe target behavior problems of the client, so that based on the inputted target behavior problems, a questionnaire for the user to complete is generated. Then, based on the response to the questionnaire, the probable function(s) of the target behavior problem of the client are determined. Using the generative AI model based on prompts that are designed based on related empirical theories and practical experiences, the response to the questionnaire, the probable function(s) of the target behavior problem and the logic of the competing behavior pathway model, effective teaching strategies and lesson plans are generated for the user to reference and apply to the teaching of the client. In addition to assisting the user (e.g., special education personnel) in collecting information more easily in order to assess behavior problems of the client, by automatically generating a corresponding strategy and automatically formulating a lesson plan that better meets the needs of the client for the user to reference and edit, the disclosure alleviates the current problems of shortage and high turnover rate of special education personnel and poor personnel training, increases efficiency in the development of lesson plans, and thereby achieves the object of this disclosure.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for administering a functional behavior assessment (FBA) and generating a positive behavior support (PBS) strategy, the method being implemented by a computer device and comprising steps of:
displaying a behavior problem collection page for inputting of a target behavior problem of a client;
based on the target behavior problem thus inputted, generating and displaying a questionnaire to be completed;
based on a response to the questionnaire, determining a probable function of the target behavior problem of the client;
based on the response to the questionnaire, the probable function of the target behavior problem, and logic of a competing behavior pathway model, generating and displaying at least one PBS strategy for selection; and
based on a selected PBS strategy of the at least one PBS strategy, generating and displaying a lesson plan that corresponds to the selected PBS strategy.

2. The method as claimed in claim 1, wherein the step of displaying a behavior problem collection page includes:
the behavior problem collection page displaying a plurality of behavior problems for selection, and displaying an input field for manual entry of a target behavior problem; and
the computer device making the target behavior problem thus manually entered serve as one of selected behavior problems, and enabling the selected behavior problems to be dragged and reordered according to severity levels thereof and to serve as the target behavior problem thus inputted.

3. The method as claimed in claim 1, wherein, in a case where the computer device determines that a reply to a questionnaire question about the reason for engaging in a target behavior problem is inputted as unsure, the step of generating and displaying a questionnaire to be completed includes:
the computer device further displaying on the questionnaire a first behavior evaluation form and a second behavior evaluation form to be completed; and
the computer device automatically performing a cross-comparison on a response to the first behavior evaluation form and a response to the second behavior evaluation form to determine the probable function of the target behavior problem.

4. The method as claimed in claim 3, wherein performing a cross-comparison is to utilize a decision-tree algorithm to perform the cross-comparison to determine the probable function of the target behavior problem, the determination being made based on foundational principles including:
a. in a case where the response to the first behavior evaluation form and the response to the second behavior evaluation form indicate a same function of the target behavior problem, giving the function of the target behavior problem a highest rating, and if response rates to questions related to the function of the target behavior problem on the first behavior evaluation form and on the second behavior evaluation form exceed 50%, determining the function of the target behavior problem with the highest rating to be the probable function of the target behavior problem of the client;
b. in a case where the response to the first behavior evaluation form and the response to the second behavior evaluation form indicate multiple functions of the target behavior problem, and the response rates to questions related to two of the multiple functions of the target behavior problem on the first behavior evaluation form and on the second behavior evaluation form exceed 50%, determining the two of the multiple functions of the target behavior problem to be the probable functions of the target behavior problem of the client; and
c. in a case other than the abovementioned principles a. and b., recommending that the first behavior evaluation form and the second behavior evaluation form be completed again, and performing a cross-comparison on a new response to the first behavior evaluation form and a new response to the second behavior evaluation form.

5. The method as claimed in claim 1, wherein the step of generating and displaying at least one PBS strategy for selection includes:
generating a strategy generation page that includes the at least one PBS strategy, the strategy generation page including, with respect to each of the at least one PBS strategy, a field that displays contents of the PBS strategy, and an edit button to be selected, the strategy generation page further including a save strategy button to be selected;
in response to the edit button being selected, allowing to be edited the field that displays the contents of the PBS strategy;
in response to the save strategy button being selected, saving the contents of the PBS strategy and generating a strategy selection page that includes the at least one PBS strategy and a generate plan button; and
in response to one of the at least one PBS strategy on the strategy selection page being selected as the selected PBS strategy, displaying on the strategy selection page the contents of the selected PBS strategy; and
wherein the step of generating and displaying a lesson plan includes:
in response to the generate plan button on the strategy selection page being selected, generating the lesson plan based on the contents of the selected PBS strategy.

6. The method as claimed in claim 1, the lesson plan including a curriculum plan, a list of activity steps and activity times corresponding to the activity steps, respectively, a modify button, an undo button, a save button, and a regenerate plan button, the method further comprising steps of:
in response to the modify button being selected, allowing to be edited the curriculum plan, the list of activity steps and the corresponding activity times;
in response to the undo button being selected, restoring to an unmodified version the curriculum plan, the list of activity steps and the corresponding activity times thus edited;
in response to the save button being selected, saving the curriculum plan, the list of activity steps and the corresponding activity times of the lesson plan; and
in response to the regenerate plan button being selected, regenerating a lesson plan based on the contents of the selected PBS strategy.

7. The method as claimed in claim 1, wherein the computer device, utilizing a generative artificial intelligence (AI) model based on a first prompt, the response to the questionnaire, the probable function of the target behavior problem, and the logic of the competing behavior pathway model, generates the at least one PBS strategy; and the computer device, utilizing the generative AI model based on a second prompt and contents of the selected PBS strategy, generates the lesson plan.

8. A device for administering a functional behavior assessment (FBA) and generating a positive behavior support (PBS) strategy, comprising:
a display unit;
an input unit; and
a processing unit electrically connected to said display unit and said input unit, wherein said processing unit is configured to:
make said display unit display a behavior problem collection page for inputting of a target behavior problem of a client via said input unit;
based on the target behavior problem thus inputted, generate and make said display unit display a questionnaire to be completed via said input unit, wherein the questionnaire includes detailed questions related to the target behavior problem thus inputted;
based on a response to the questionnaire, determine a probable function of the target behavior problem of the client;
based on the response to the questionnaire, the probable function of the target behavior problem and logic of a competing behavior pathway model, generate at least one PBS strategy and make said display unit display the at least one PBS strategy for selection via said input unit; and
based on a selected PBS strategy of the at least one PBS strategy, generate a lesson plan that corresponds to the selected PBS strategy and make said display unit display the lesson plan.

9. The device as claimed in claim 8, wherein:
the behavior problem collection page displays a plurality of behavior problems for selection via said input unit, and displays an input field for manual entry of a target behavior problem; and
said processing unit is configured to make the target behavior problem thus manually entered serve as one of selected behavior problems, and enable the selected behavior problems to be dragged and reordered according to severity levels thereof and to serve as the target behavior problems thus inputted.

10. The device as claimed in claim 8, wherein, in a case where said processing unit determines that a reply to a questionnaire question about the reason for engaging in a target behavior problem is inputted as unsure, said processing unit is configured to:
make the questionnaire further include a first behavior evaluation form and a second behavior evaluation form to be completed; and
automatically perform a cross-comparison on a response to the first behavior evaluation form and a response to the second behavior evaluation form to determine the probable function of the target behavior problem.

11. The device as claimed in claim 10, wherein said processing unit is configured to perform the cross-comparison by utilizing a decision-tree algorithm to determine the probable function of the target behavior problem, the determination being made based on foundational principles including:
a. in a case where the response to the first behavior evaluation form and the response to the second behavior evaluation form indicate a same function of the target behavior problem, giving the function of the target behavior problem a highest rating, and if response rates to questions related to the function of the behavior problem on the first behavior evaluation form and on the second behavior evaluation form exceed 50%, determining the function of the target behavior problem with the highest rating to be the probable function of the target behavior problem of the client;
b. in a case where the response to the first behavior evaluation form and the response to the second behavior evaluation form indicate multiple functions of behavior, and the response rates to questions related to two of the multiple functions of behavior on the first behavior evaluation form and on the second behavior evaluation form exceed 50%, determining the two of the multiple functions of behavior to be the probable functions of the target behavior problem of the client; and
c. in a case other than the abovementioned principles a. and b., recommending that the first behavior evaluation form and the second behavior evaluation form be completed again, and performing a cross-comparison on a new response to the first behavior evaluation form and a new response to the second behavior evaluation form.

12. The device as claimed in claim 8, wherein:
said processing unit is configured to generate a strategy generation page that includes the at least one PBS strategy, and make said display unit display the strategy generation page;
the strategy generation page includes, with respect to each of the at least one PBS strategy, a field that displays contents of the PBS strategy, and an edit button to be selected, the strategy generation page further including a save strategy button to be selected;
in response to the edit button being selected, said processing unit is configured to allow to be edited via said input unit the field that displays the contents of the PBS strategy;
in response to said processing unit determining that the save strategy button is selected, said processing unit is configured to save the contents of the PBS strategy, generate a strategy selection page that includes the at least one PBS strategy and a generate plan button, and make said display unit display the strategy selection page;
in response to one of the at least one PBS strategy on the strategy selection page being selected as the selected PBS strategy, said processing unit is configured to make said display unit display on the strategy selection page the contents of the selected PBS strategy; and
in response to the generate plan button on the strategy selection page being selected, said processing unit is configured to generate the lesson plan based on the contents of the selected PBS strategy.

13. The device as claimed in claim 8, wherein:
the lesson plan includes a curriculum plan, a list of activity steps and activity times corresponding to the activity steps, respectively, a modify button, an undo button, a save button, and a regenerate plan button;
in response to the modify button being selected, said processing unit is configured to allow to be edited the curriculum plan, the list of activity steps and the corresponding activity times;
in response to the undo button being selected, said processing unit is configured to restore to an unmodified version the curriculum plan, the list of activity steps and the corresponding activity times thus edited;
in response to the save button being selected, said processing unit is configured to save the curriculum plan, the list of activity steps and the corresponding activity times of the lesson plan; and
in response to the regenerate plan button being selected, said processing unit is configured to regenerate a lesson plan based on the contents of the selected PBS strategy.

14. The device as claimed in claim 8, wherein said processing unit is configured to:
utilize a generative artificial intelligence (AI) model based on a first prompt, the response to the questionnaire, the probable function of the target behavior problem, and the logic of the competing behavior pathway model to generate the at least one PBS strategy; and
utilize the generative AI model based on a second prompt and contents of the selected PBS strategy to generate the lesson plan.

15. A non-transitory computer-readable recording medium storing a software program that, when installed and executed by a computer device, enables the computer device to implement the method as claimed in claim 1.
